(19) United States
(12) Patent Application Publication
(10) Pub. No.: US 2007/0197426 A1
(43) Pub. Date: Aug. 23, 2007

(54) Lactoferrin polypeptide, process for producing the same and inflammation inducing substance

(76) Inventors: Kenichi Komine, Miyagi (JP); Shunji Sugawara, Miyagi (JP)

Correspondence Address: YOUNG & THOMPSON, 745 SOUTH 23RD STREET, 2ND FLOOR, ARLINGTON, VA, 22202, US

(21) Appl. No.: 10/589,482

(22) PCT Filed: Feb. 16, 2004
(86) PCT No.: PCT/JP04/01614

§ 371 Date: Oct. 30, 2006

(51) Int. Cl. A61K 38/40; C12P 21/06; C07K 14/79

(52) U.S. Cl. 514/6; 530/400; 435/68.1

(57) ABSTRACT

To provide lactoferrin polypeptides which have inducing activity for production of various inflammatory cytokines and various chemokines. Lactoferrin polypeptides characterized by comprising the amino acid sequence of phenylalanine (F), lysine (K) and aspartic acid (D). They are obtained by digestion with proteases. Molecular weights of them are less than 25 kDa.

### TECHNICAL FIELD

The present invention is an invention relating to new polypeptides contained in a lactoferrin molecule having inflammatory inducing effects such as inducing abilities for inflammatory cytokine production and chemokine production.

### BACKGROUND ART

(Patent Literature 1) JP, 2003-289749, A

(Patent Literature 2) JP, 2004-002471, A

(Non-patent Literature 1) Annu. Rev. Nutr. 1995. 15. 93-110, Pediatr. Res. 1996. 40. 257-262, J. Peptide Res. 2001. 57. 240-249, J. Vet. Med. Sci. 2002. 64. 873-878.

(Non-patent Literature 2) FEMS Microbiol. Lett. 1996. 145. 209-214, Pro. Natl. Acad. Sci. USA. 1998. 95, 12641-12646. Mol. Microbiol. 2003. 47. 607-617.

Lactoferrin is a multifunctional glycoprotein, which will increase in body fluid such as milk, saliva, tear fluid, feces, urine, blood and the like under various diseases. Many useful effects for organism such as diseases. Many useful effects for organism such as antibacterial activity, anti-virus activity, lymphocyte activating effect, anti-tumor effect and iron affinity have been reported on it (the non-patent literature 1: Annu. Rev. Nutr. 1995. 15. 93-110, Pediatr. Res. 1996. 40. 257-262, J. Peptide Res. 2001. 57. 240-249, J. Vet. Med. Sci. 2002. 64. 873-878). Therefore, many researches have been performed to apply lactoferrin to various diseases. On the other hand, it is thought for the lactoferrin increased in bacterial infectious diseases that their effects are lost and inactivated by digestion with enzymes produced by bacterium (the non-patent literature 2: FEMS Microbiol. Lett. 1996. 145. 209-214, Pro. Natl. Acad. Sci. USA. 1998. 95, 12641-12646. Mol. Microbiol. 2003. 47. 607-617). Consequently, inflammatory symptom is thought to be aggravated in spite of presence of lactoferrin. In bovine mastitis, a bovine bacterial infectious disease, it has been reported that a MW 30 to 60 kDa lactoferrin protein group referred to as “inflammatory lactoferrin” which exhibit inflammatory effect in milk increases and aggravates inflammation (the patent literature 1; JP, 2003-289749, A). However, for this inflammatory lactoferrin protein group, detailed site and structure exhibiting the inflammatory effect have not been revealed and there are many unknown points in their properties and physiological action mechanisms.

On the other hand, a following peptide is described in the patent literature 2 (JP, 2004-002471, A).

It is a peptide consisting of a amino acid sequence presented by the amino acid sequence: “Ala-Pro-Arg-Lys-Asn-Val-Arg-Trp-Cys-Thr-Ile-Ser-Gln-Pro-Asp-Ser-Phe-Lys”. In addition, a technology for digesting bovine lactoferrin with proteases and obtaining the peptide above from enzyme digestives.

Exemplary enzymes of which amount in body fluid increase in inflammatory disease include the elastase produced by white blood cell, one of serine-proteases (Clinica, Chemica. Acta. 1995. 239, 91-101). Elastase is known to degrade complement components and globulin at inflammation (Am. J. Pathol. 1979. 94. 75-83, Elastase. (R. P. Mecham, ed) 1986. Catalytic and biological properties. In: Biological of Extracellular Matrix Orlando, Fla.: Academic Press, 217-320, Biochemistry. 1997. 16. 3390-3396). However, there is no case reporting involvement of this elastase and lactoferrin. And, for the lactoferrin being digested with bacteria produced enzymes in bacterial diseases, its physiological action have not been reported.

Peptide described in the patent literature 2 is an immune activator but not have inflammatory inducing activity.

In the present invention, with having views upon the elastase which increase in inflammatory diseases, it is demonstrated that lactoferrin can be digested and it makes possible to isolate polypeptides having inducing activity for production of various inflammatory cytokines and chemokines from digested lactoferrin. And a new lactoferrin polypeptide having such inflammatory inducing activity is found from these polypeptides by synthesizing polypeptides having amino acid sequences present in human lactoferrin. The present invention has been made based on this new finding.

An object of the present invention intends to provide lactoferrin polypeptides having inflammatory inducing effect.

The present invention intends to provide inflammatory inducing substances.

The present invention intends to provide production methods for isolating and purifying lactoferrin polypeptides having inflammatory inducing effect from saliva. It also intends to provide methods for producing synthetic peptide thereof.

### DISCLOSURE OF THE INVENTION

The lactoferrin polypeptide of the present invention is characterized by comprising the amino acid sequence of phenylalanine (F), lysine (K) and aspartic acid (D).

The lactoferrin polypeptide is preferably less than MW 25 kDa.

Preferably, the lactoferrin polypeptide is obtained by digesting human lactoferrin with protease.

The inflammatory inducing substance of the present invention is characterized in that it is based on said lactoferrin polypeptide having inducing activity for production of various inflammatory cytokines, and synthetic peptides thereof.

The inflammatory inducing substance of the present invention is characterized in that it is based on said lactoferrin polypeptide having inducing activity for production of various inflammatory chemokines, and synthetic peptides thereof.

The inflammatory inducing substance of the present invention is characterized in that it is based on said lactoferrin polypeptide having enhancing effect for expression of NFκB, an intracellular transcription factor inducing productions of cytokines and chemokines, and synthetic peptide thereof.

The production method for producing the synthetic peptide of the present invention is characterized in that the synthetic peptide is prepared by digesting human or bovine lactoferrin with protease, purifying it by SDS-polyacrylamide gel electrophoresis, gel filtration, concanavalin A (Con A) affinity chromatography and lactoferrin antibody attached affinity chromatography, isolating lactoferrin polypeptide comprising the amino acid sequence of phenylalanine (F), lysine (K) and aspartic acid (D), and determining it by amino acid sequencer.

In the present invention, lactoferrin was purified by concentrating saliva from human affected by inflammatory disease (periodontal disease) with 50% saturated ammonium sulfate precipitation method, desalting it with Tris-HCl buffer, and then applying it to human-lactoferrin antibody (ICN Pharmaceuticals, Inc., USA) CNBr activated Sepharose 4B (Pharmacia, Sweden). Furthermore, human lactoferrin (ICN Pharmaceuticals, Inc., USA) was treated with elastase (SIGMA, USA), one kind of metalloprotease, at 37° C. for 1 hr, and then reaction was stopped with elastase inhibitor (CALBIOCHEM-NOVABIOCHEM, Inc., USA). Lactoferrin obtained from inflammatory disease samples and lactoferrin treated with elastase were subjected to concanavalin A (Con A) two-dimensional immunoelectrophoresis and their electropherograms were observed. In addition, human lactoferrin from inflammatory disease sample and human lactoferrin treated with elastase were subjected to SDS-polyacrylamide electrophoresis (SDS PAGE) using 15% polyacrylamide gel. After electrophoresis, it was transferred to PVDF membrane (BioRad, USA) and bands being common among each sample were cut off, and then their N-terminal amino acid sequences were analyzed by an amino acid sequencer (Hewlett Packard, USA). Then, each lactoferrin polypeptide was prepared and their inducing abilities for cytokine and chemokine productions were analyzed on various cells to identify peptides having inflammatory inducing activity.

The amino acid sequence described in the patent literature 2 is different from the amino acid sequence according to the present invention.

The amino acid sequence according to the present invention is a peptide containing Phe-Lys-Asp (abbreviation code: FKD). And the location of N-amino acid sequence is close to C-terminal region. Furthermore, FKD exists in two locations for human lactoferrin and only one location for bovine lactoferrin.

Therefore, a lactoferrin polypeptide containing this FKD is the entity exhibiting inflammatory inducing effect.

According to the present invention, new lactoferrin polypeptides having the amino acid sequence (FKD) are substances having inflammatory effect, which can be referred to as “inflammatory inducing lactoferrin-polypeptide”. A discovery of domain having inflammatory effect in addition to previously known useful physiological effect of lactoferrin could contribute to a great advance of may research area including effective use of lactoferrin, inflammatory disease and various infectious diseases.

### BRIEF DESCRIPTION OF DRAWINGS

FIG. 1 is electrophoretic patterns of SDS PAGE, western blotting and Con A two-dimensional electrophoresis of lactoferrin from periodontitis patients saliva and elastase treated human lactoferrin.

FIG. 2 shows changes of molecular weight and electrophoretic pattern of Con A two-dimensional electrophoresis of human lactoferrin after elastase treatment.

FIG. 3 shows the molecular weights, the amino acid sequences of N-terminals and the locations within lactoferrin molecule.

FIG. 4 shows inflammatory cytokines, chemokines and NFκBp65 expression enhancing effect by co-culture with human-lactoferrin-polypeptide produced.

### BEST MODE FOR CARRYING OUT THE INVENTION

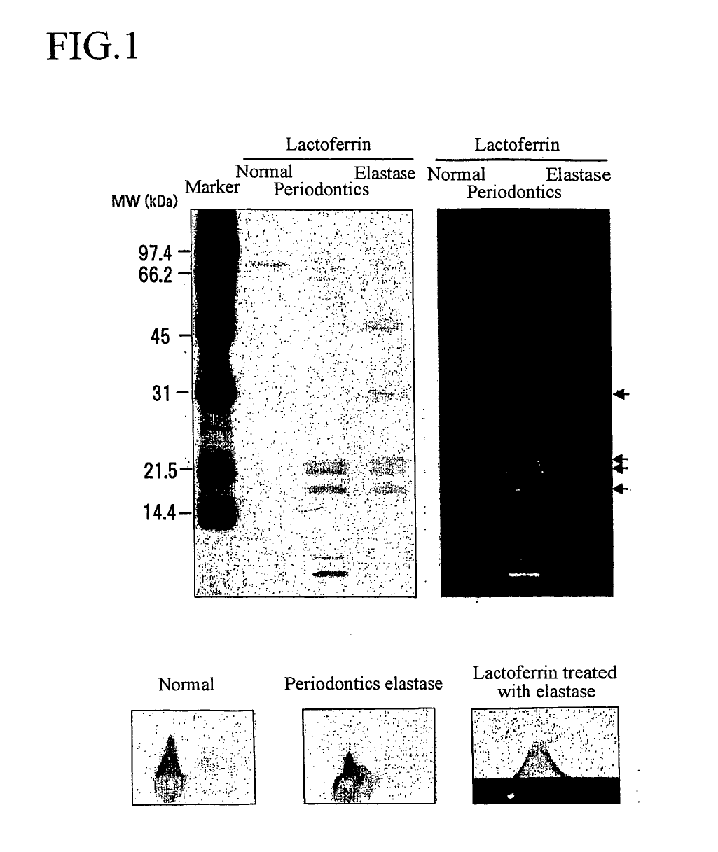

For human samples from inflammatory disease and samples treated with elastase, it was confirmed that lactoferrin in the samples had been downsized. And, from Con A two-dimensional electrophoreogram, it was found that fraction having low Con A affinity was emerged in both samples. It is shown in FIG. 1.

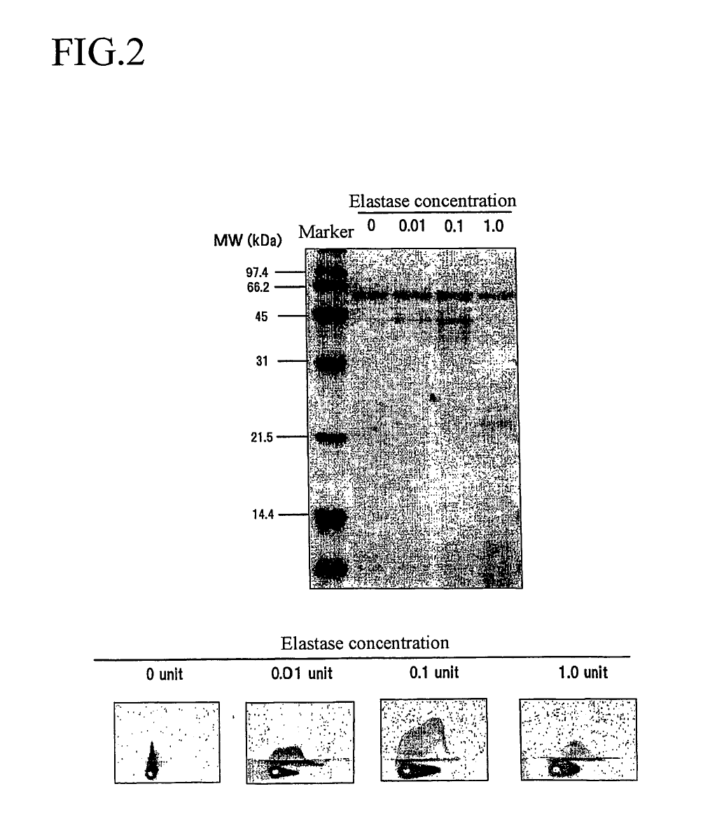

Reaction was performed with changing elastase concentration stepwise from 0.001 unit to 1.0 unit, and it resulted in increase of small molecule lactoferrin with around 20 kDa of molecular weight with increase of elastase reaction concentration. Furthermore, increase in low Con A affinity fraction was also observed in Con A two-dimensional electrophoresis. Such fragmented lactoferrin was clearly different from “inflammatory lactoferrin” protein group (molecular weight 30 to 60 kDa) that had been reported for bovine mastitis milk. It is shown in FIG. 2.

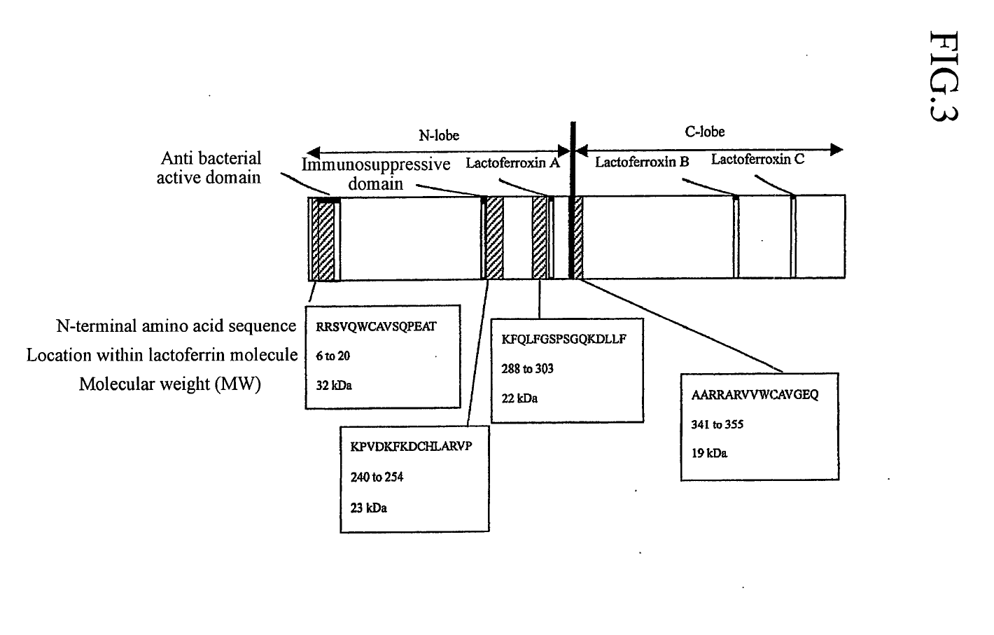

Lactoferrin domains that were commonly found in human samples from inflammatory disease and human samples treated with elastase are as shown in FIG. 1. Among them, a domain, which is at N-terminal side in human and bovine, contains the amino acid sequence being reported to have anti bacterial activity and apoptosis activity against tumor cells. Among human domains, a domain located halfway in N-terminal amino acid sequence has a part of the mini-domain that has immunosuppressive activity. Two other domains located downstream from it were not reported ones. It is shown in FIG. 3.

Amino acid sequence analysis showed that among these lactoferrin domains, excluding domains of which function had been already reported, two main lactoferrin molecules of MW 20 to 25 kDa, commonly found in human lactoferrin treated with elastase and lactoferrin in inflammatory disease secretion, were located in N-terminal region. Then, peptides were prepared from these two domains and their physiological effects were analyzed. Prepared polypeptides are as shown in table 1.

TABLE 1
List of prepared human lactoferrin polypeptide

| Prepared | Amino acid | Location in human |
|---|---|---|
| Peptide | sequence | lactoferrin molecule |
| HuPep1. | FKDCHLA | 243 to 249 |
| HuPep2. | VPSHAVVAR | 251 to 259 |
| HuPep3. | FQLFGSP | 287 to 293 |
| HyPep4. | GQKDLLFKDSAI | 295 to 307 |

Lymphocytes were separated from normal human peripheral blood, which was collected by using heparin sodium, by specific gravity centrifugation using Lympholight H (Cedarlane, Canada), and inducing ability for production of various cytokines and chemokines and inducing ability for expression of NFκBp65, a cellular transcription factor, were analyzed using the lymphocytes. Human cytokines (IL-6 and TNFα; Techne Co., USA), chemokines (IL-8 and MCP-1; American Research Product, Inc., USA) and on NFκB (TransAM NFκB family transcription factor assay kit; Active Motif, Co., USA) were measured by enzyme antibody method.

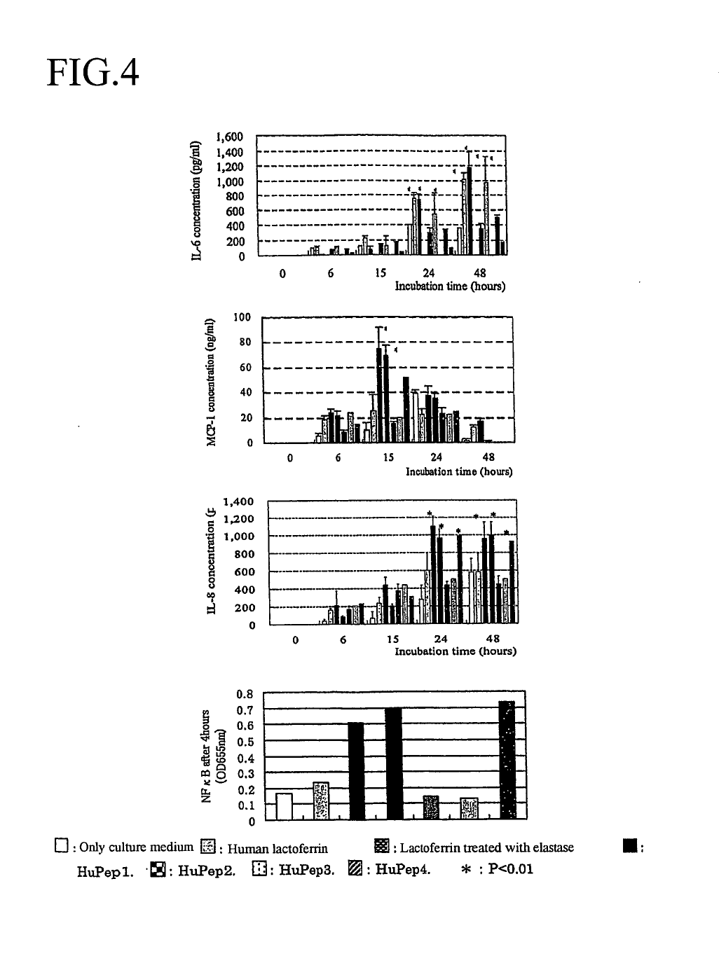

Among polypeptides isolated and prepared from human lactoferrin treated with elastase, production of IL-6, one inflammatory cytokine, could be confirmed on HuPep1 and HuPep4, as shown in FIG. 2. Furthermore, inducing abilities for production of IL-8 and MCP-1, which increased in blood at inflammation, and enhancement of expression of NFκBp65, a cellular transcription factor responsible for induction of these cytokines and chemokines production, were also confirmed for these polypeptides. Amino acid sequence FKD is common between HuPep1 and HuPep 4. It is shown in FIG. 4.

For human, new lactoferrin polypeptide found in the present invention, which is contained in human lactoferrin digested by elastase and comprise amino acid sequence of FKD, is found in two places. And this new lactoferrin polypeptide is a substance that enhances expression of NFκB, a cellular transcription factor, induces production of cytokines having cytotoxic effect etc., as well as production of chemokines having lymphocyte chemotactic activity, and causes inflammation. Furthermore, it is known that NFκB induces production of inflammatory mediators such as nitric oxide (NO), arachidonic acid metabolites and various enzymes. Thus, a new lactoferrin polypeptide found in the present invention is the new lactoferrin polypeptide on which it is easily expected to have an ability to induce production of these inflammatory mediators. Furthermore, it is likely that digestion with protease other than elastase produces the lactoferrin molecule. The amino acid sequence comprising FKD is also present at one place in bovine lactoferrin (from site 300 to 302 from N-terminal), and it locates a similar site where the polypeptide can be found in human lactoferrin. And amino acids to be digested with protease such as elastase are located near it. Therefore, it is expected that these proteases can digest lactoferrin. That is, the new lactoferrin-polypeptide having the same inflammatory inducing effect can be also separated from lactoferrin having the same amino acid sequence (FKD) that is derived from non-human animals source such as bovine.

### INDUSTRIAL APPLICABILITY

According to the present invention, the new lactoferrin-polypeptide having the amino acid sequence (FKD) is an inflammatory inducing substance, which can be referred to as “inflammatory inducing lactoferrin-polypeptide”. According to the present invention, in addition to known useful physiological effects of lactoferrin, the discovery of the domain having inflammatory inducing effect would make great contribution to advance in many research areas such as utilization of lactoferrin, inflammatory diseases and various infectious diseases.

1-11. (canceled)

12. A lactoferrin polypeptide comprising the amino acid sequence of phenylalanine (F), lysine (K) and aspartic acid (D) obtained by treating lactoferrin with serine protease.

13. The lactoferrin polypeptide according to claim 12 wherein the serine protease is elastase.

14. The lactoferrin polypeptide according to claim 12 characterized in that its molecular weight is less than 25 kDa.

15. The lactoferrin polypeptide according to claim 13 characterized in that its molecular weight is less than 25 kDa.

16. Inflammatory inducing substances based on a lactoferrin polypeptide which has inducing activity for production of various inflammatory cytokines and comprise the amino acid sequence of phenylalanine (F), lysine (K) and aspartic acid (D), and on the synthetic polypeptide thereof.

17. Inflammatory inducing substances based on a lactoferrin polypeptide which has inducing activity for production of various chemokines and comprise the amino acid sequence of phenylalanine (F), lysine (K) and aspartic acid (D), and on the synthetic polypeptide thereof.

18. Inflammatory inducing substance based on a lactoferrin polypeptide which has enhancing activity for expression of NFκB and comprise the amino acid sequence of phenylalanine (F), lysine (K) and aspartic acid (D), and on the synthetic polypeptide thereof.

19. Inflammatory inducing substances based on a lactoferrin polypeptide which comprises the amino acid sequence of phenylalanine (F), lysine (K) and aspartic acid (D), and on the synthetic polypeptide thereof.

20. The inflammatory inducing substance according to claim 16 wherein its molecular weight is less than 25 kDa.

21. The inflammatory inducing substance according to claim 16 characterized in that it is obtained by treating lactoferrin with serine protease.

22. The inflammatory inducing substance according to claim 21 wherein the serine protease is elastase.

23. A production method for isolating and purifying a lactoferrin polypeptide comprising the amino acid sequence of phenylalanine (F), lysine (K) and aspartic acid (D) from human or bovine lactoferrin by digesting the human or the bovine lactoferrin with serine protease, followed by purification.

24. The production method according to claim 23 wherein the serine protease is elastase.

25. A synthetic peptide production method characterized by determining the lactoferrin polypeptide according to claim 23 with amino acid sequencer and preparing a synthetic peptide.

26. The production method according to claim 23 for the synthetic peptide characterized in that said purification is carried out by SDS-polyacrylamide gel electrophoresis, gel filtration, concanavalin A (Con A) affinity chromatography and lactoferrin antibody attaching affinity chromatography.
